(19) United States
(12) Patent Application Publication
(10) Pub. No.: US 2025/0302420 A1
(43) Pub. Date: Oct. 2, 2025

(54) METHOD AND APPARATUS FOR PROVIDING INFORMATION FOR PREDICTING MEDIASTINAL LYMPH NODE METASTASIS OF LUNG CANCER

(71) Applicant: NATIONAL CANCER CENTER, Goyang-si (KR)

(72) Inventors: Bin HWANGBO, Goyang-si (KR); Eun Young PARK, Goyang-si (KR); Goyang-si LEE, Goyang-si (KR); Hyun Sung CHUNG, Goyang-si (KR)

(73) Assignee: NATIONAL CANCER CENTER, Goyang-si (KR)

(21) Appl. No.: 19/098,192

(22) Filed: Apr. 2, 2025

(30) Foreign Application Priority Data
Apr. 2, 2024 (KR) 10-2024-0044755

(51) Int. Cl. A61B 6/00; G16H 50/30

(52) U.S. Cl. CPC A61B 6/5294; A61B 6/5235; G16H 50/30

(57) ABSTRACT

The present disclosure relates to a method and apparatus for predicting mediastinal lymph node metastasis in non-small cell lung cancer based on information regarding a patient's age, a histological type of a tumor, a location of the tumor, a size of the tumor, a clinical lymph node stage determined by CT, and a clinical lymph node stage determined by PET-CT. According to an aspect of the present disclosure, a method and apparatus for predicting mediastinal lymph node metastasis in non-small cell lung cancer provides information on the presence of mediastinal lymph node metastasis in potentially operable lung cancer patients, specifically non-small cell lung cancer patients, and thus can be usefully used for decision-making on staging and treatment methods for non-small cell lung cancer patients, such as invasive mediastinal staging.

### CROSS-REFERENCE TO RELATED APPLICATION

This application is based on and claims priority under 35 U.S.C. § 119 to Korean Patent Application No. 10-2024-0044755, filed on Apr. 2, 2024, in the Korean Intellectual Property Office, the disclosure of which is incorporated by reference herein in its entirety.

### BACKGROUND

### 1. Field

The present disclosure relates to a method and apparatus for predicting mediastinal lymph node metastasis of lung cancer.

### 2. Description of the Related Art

The treatment method of lung cancer varies depending on the stage of cancer progression at the time of diagnosis. Surgery is possible in cases with a low stage, but surgery is not recommended in cases where the stage has progressed significantly. Therefore, determining the clinical stage through various tests, such as imaging tests like CT or PET-CT, upon lung cancer diagnosis is important for determining the appropriate treatment method of lung cancer. The clinical N (Node) stage (cN1, cN2, cN3) classifies the extent of lung cancer metastasis to the lymph nodes before surgery.

Lung cancer is largely classified into small cell lung cancer (SCLC) and non-small cell lung cancer (NSCLC) according to histological type. In non-small cell lung cancer (NSCLC), if the disease has progressed due to mediastinal lymph node metastasis, surgery is generally not considered as the primary treatment. Therefore, invasive mediastinal staging, which involves directly obtaining lymph node tissue for histological examination, is important in determining the treatment plan for patients with NSCLC. Currently, endobronchial ultrasound-guided transbronchial needle aspiration (EBUS-TBNA), which involves performing a histological examination using a fine needle while observing mediastinal lymph nodes with endobronchial ultrasound, is the primary method of invasive mediastinal staging.

Practice guidelines for mediastinal staging in patients with non-small cell lung cancer (NSCLC) recommend performing invasive staging only for specific risk groups suspected of having mediastinal lymph node metastasis. According to the guidelines of the European Society of Thoracic Surgeons (ESTS), preoperative invasive staging is recommended for patients with a central tumor location on CT or PET, a tumor size of 3 cm or more, or a clinical N stage of 1-3 (cN1-3). According to the American College of Chest Physicians (ACCP) guidelines, invasive examination is strongly recommended for patients with clinical N stage 1-3 by CT, clinical N stage 2-3 by PET, or a centrally located tumor. Invasive mediastinal staging is not recommended for patients with clinical N stage 0 by CT and PET and peripheral tumors of 3 cm or less. The National Institute for Health and Care Excellence (NICE) guidelines recommend invasive examination for patients with a clinical N stage of 1-3 (cN1-3) determined by CT or PET-CT. Younger age and adenocarcinoma histology have been reported as risk factors for mediastinal lymph node metastasis, but have not been reflected in the staging guidelines. Risk factors associated with mediastinal lymph node metastasis, such as abnormal lymph nodes visible on CT or PET-CT, tumor location, tumor size, histological type, and age, may be interrelated. However, existing guidelines do not provide probability estimates for predicting N2-3 stage based on combinations of the aforementioned risk factors.

Accordingly, the present disclosure addresses these shortcomings by providing a mediastinal lymph node metastasis prediction model for patients with potentially operable lung cancer. The present prediction models were based on a prospective cohort study of patients who were diagnosed with lung cancer stage by EBUS-TBNA and who underwent surgical treatment to confirm the surgical stage if mediastinal metastasis was not confirmed.

### SUMMARY

An object of the present disclosure is to provide a method and an apparatus for predicting mediastinal lymph node metastasis using information regarding a lung cancer patient's age, a histological type of a tumor, a location of the tumor (central, peripheral), a size of the tumor, a clinical lymph node stage determined by CT, and a clinical lymph node stage determined by PET-CT.

According to an aspect of the present disclosure, a method for predicting mediastinal lymph node metastasis in lung cancer, includes: (a) obtaining information regarding a lung cancer patient's age, a histological type of a tumor, a location of the tumor, a size of the tumor, a clinical lymph node stage determined by CT, and a clinical lymph node stage determined by PET/CT;
(b) calculating a mediastinal lymph node metastasis prediction score for each variable obtained in step (a); and
(c) calculating a probability of mediastinal lymph node metastasis in the lung cancer based on a total score, wherein the total score is a sum of the prediction scores for all variables calculated in step (b).

The term “lung cancer,” as used in the present specification, refers to a tumor originating in the lung, and means non-small cell lung cancer such as squamous cell carcinoma, adenocarcinoma, large cell carcinoma, and large cell neuroendocrine carcinoma, excluding carcinoid tumors, tumors of salivary gland origin, and small cell lung cancer, which typically do not undergo invasive mediastinal staging for surgical decision-making. Lung cancer patients were 18 years of age or older and under 80 years of age. Patients with non-small cell lung cancer who are potentially eligible for surgery were enrolled. Patients with distant metastasis, unresectable T4, mediastinal invasion or extranodal invasion, confirmed supraclavicular lymph node metastasis, or Pancoast tumor are excluded. Subsolid nodules with a solid portion of 1 cm or less and solid nodules with cT1aN0 on CT and PET-CT are also excluded. Patients who are surgically and medically inoperable were also excluded from the development of the prediction model.

As used in the present specification, the term “variable” refers to clinical or pathological information capable of predicting mediastinal lymph node metastasis in lung cancer. Specifically, “variable” may include clinical variables such as an individual's age, a histological type of a tumor, a location of the tumor, a size of the tumor, a clinical lymph node stage determined by CT, and a clinical lymph node stage determined by PET-CT.

As used in the present specification, an individual's “age” refers to the number of years the individual has lived since birth, specifically, at least 18 years and less than 80 years. More specifically, the age may be measured at the time of predicting mediastinal lymph node metastasis. In the model development, the age on the date of performing EBUS-TBNA for invasive mediastinal staging was entered. Furthermore, the age may be classified into specific ranges (e.g., less than 60 years, 60 years to less than 70 years, or 70 years or older) and reflected as a clinical variable.

As used in the present specification, “tumor histology” refers to the pathological classification of the type of tumor cell tissue obtained from an individual, specifically a patient. Specifically, the histological type is classified as adenocarcinoma, squamous cell carcinoma, or other non-small cell lung cancer. Mixed tumors containing adenocarcinoma (e.g., adenosquamous cell carcinoma) or multiple tumors may be classified as adenocarcinoma if a solid portion containing adenocarcinoma is larger than 1 cm. Squamous cell carcinoma may be limited to pure squamous cell carcinoma, either solitary or multiple. If information on the tumor histology of the lung cancer patient is not available, the probability of mediastinal lymph node metastasis in the lung cancer may be estimated based on information on the remaining five variables, namely, the patient's age, the location of the tumor, the size of the tumor, the clinical lymph node stage by CT, and the clinical lymph node stage by PET-CT.

As used in the present specification, “location of the tumor” refers to the location of the tumor as seen on imaging, and it can be generally classified as being located in the central or peripheral region of the lung and reflected as a clinical variable. Specifically, a central tumor was defined as one located in the inner one-third of the hemithorax based on the innermost part of the tumor on CT, and the lines dividing the hemithorax into thirds were drawn as concentric circles arising from the midline.

As used in the present specification, “size of the tumor” may refer to the size of the tumor as seen on imaging (axial CT scan), expressed in cm. Specifically, the tumor size is classified as 3 cm or less, greater than 3 cm and up to 5 cm, or larger than 5 cm, and is reflected as a clinical variable. Furthermore, the tumor size is reflected in the clinical tumor stage (cT) as a clinical variable. More specifically, the cT stage may be determined as cT0, cT1, cT2, cT3, or cT4, with increasing stage indicating a greater extent of a tumor according to the clinical judgment of the progression of the tumor (or cancer).

As used in the present specification, “clinical lymph node stage by CT” and “clinical lymph node stage by PET-CT” each refer to clinical N stage (cN). Specifically, the cN stage is determined as cN0, cN1, cN2, or cN3, with increasing stage indicating a greater extent of metastasis according to the clinical judgment of the progression of the tumor metastasis. Specifically, the clinical lymph node stage (cN) by CT and the clinical lymph node stage (cN) by PET-CT may be different. The positive criterion for CT lymph nodes is defined as a lymph node short axis diameter of 1 cm or more, and the positive criterion for PET-CT lymph nodes is increased uptake of fluorodeoxyglucose F18 compared to mediastinal blood flow.

Of the above variables, tumor location, tumor size, clinical lymph node stage by CT, and/or clinical lymph node stage by PET-CT are analyzed based on radiological images from CT or PET-CT. However, the CT and PET-CT are analyzed if performed within 30 days prior to the date of performing EBUS-TBNA.

In an embodiment, the clinical variables may be measured at the time of diagnosis of lung cancer, specifically non-small cell lung cancer, at the time of prediction or diagnosis of mediastinal lymph node metastasis, or at the time of invasive mediastinal staging.

In an embodiment, in step (b) of calculating the mediastinal lymph node metastasis prediction score, a nomogram may be used, wherein information for each variable is matched to at least a portion of a score line having a minimum value and a maximum value.

As used in the present specification, “nomogram” refers to a visualization of a multivariable logistic model constructed to predict mediastinal lymph node metastasis in an individual based on information regarding the patient's age, the histological type of the tumor, the location of the tumor, the size of the tumor, the clinical lymph node stage by CT, and the clinical lymph node stage by PET-CT. Specifically, it is configured to probabilistically calculate the presence or absence of mediastinal lymph node metastasis in an individual, ultimately based on each of the levels of variables selected from the group consisting of patient age, tumor histology, tumor location, tumor size, and clinical lymph node stage by CT or PET-CT. A prediction score for each variable is calculated using the prediction model, and the prediction scores for each variable are summed, and based on this, the presence or absence of lymph node metastasis in the individual is visualized as a probability.

In an embodiment, in step (b) of calculating the mediastinal lymph node metastasis prediction score and step (c) of calculating the probability of mediastinal lymph node metastasis in lung cancer, the mediastinal lymph node metastasis prediction score may be calculated according to a PLUS-M model nomogram, a PLUS-E model nomogram, or both, shown below.

In an embodiment, step (b) may include fitting a multivariable logistic model to the variables obtained in step (a) to calculate a linear predictor value using regression coefficient values for each variable, and calculating a prediction score for each variable. Step (c) may include calculating a probability of mediastinal lymph node metastasis in the lung cancer from a total score which is a sum of the prediction scores for all variables calculated in step (b).

Further, the present disclosure provides a method for predicting mediastinal lymph node metastasis in lung cancer, the method including: (a-1) obtaining information regarding a lung cancer patient's age, a histological type of a tumor, a location of the tumor, a size of the tumor, a clinical lymph node stage determined by CT, and a clinical lymph node stage determined by PET-CT; and
(b-1) deriving a probability of mediastinal lymph node metastasis in the lung cancer based on the information obtained in step (a-1), according to Equation 1, Equation 2, or both, shown below:

$$ln ( \frac{p}{1 - p} ) = - 5.2087 + 1.39 \; 18 \times I ( Age < 60 ) + 0.8888 \times I ( 60 \leq Age < 70 ) + 1.3 \; 945 \times I ( Histology = Adenocarcinoma ) + 1.3489 \times I ( Histology = Other non - squamous carcinoma ) + 0.517 \times I ( Location = central ) + 0.8184 \times I ( 3 cm < Tumor size \leq 5 cm ) + 0.4647 \times I ( Tumor size > 5 cm ) + 1.224 \times I ( cN by CT = N 1 ) + 1.74 \; 89 \times I ( cN by CT = N 2 or 3 ) + 1.6629 \times I ( cN by PET / CT = N 1 ) + 2.4 \; 198 \times I ( cN by PET / CT = N 2 or 3 ) \qquad [ Equation 1 ]$$

$$ln ( \frac{p}{1 - p} ) = - 5.4838 + 1.25 \; 38 \times I ( Age < 60 ) + 0.6143 \times I ( 60 \leq Age < 70 ) + 1.3 \; 333 \times I ( Histology = Adenocarcinoma ) + 1.0183 \times I ( Histology = Other non - squamous carcinoma ) + 0.3006 \times I ( Location = central ) + 0.7679 \times I ( 3 cm < Tumor size \leq 5 cm ) + 0.4159 \times I ( Tumor size > 5 cm ) + 1.2494 \times I ( cN by CT = N 1 ) + 2.02 \; 04 \times I ( cN by CT = N 2 or 3 ) + 1.7475 \times I ( cN by PET / CT = N 1 ) + 2.5 \; 994 \times I ( cN by PET / CT = N 2 or 3 ) \qquad [ Equation 2 ]$$

In the above equations, p represents a predicted probability of mediastinal lymph node metastasis (N2-3), and I represents an indicator variable for an individual clinical factor.

The descriptions of lung cancer and the variables (lung cancer patient's age, histological type of the tumor, location of the tumor, size of the tumor, clinical lymph node stage by CT, and clinical lymph node stage by PET-CT) are as described above.

The mediastinal lymph node metastasis prevalence prediction model according to Equation 1 may be referred to as PLUS-M. Furthermore, the mediastinal lymph node metastasis diagnostic prediction model according to Equation 2 may be referred to as PLUS-E. PLUS-M is an abbreviation for Prediction model for Lung cancer Staging-Mediastinal metastasis, and is a model for predicting mediastinal lymph node metastasis, including EBUS-TBNA and surgery. PLUS-E is an abbreviation for Prediction model for Lung cancer Staging-mediastinal metastasis detection by EBUS-TBNA, and is a prediction model for diagnosing mediastinal lymph node metastasis by EBUS-TBNA. The method may be configured to provide information on the presence or absence of mediastinal lymph node metastasis in lung cancer based on information regarding the patient's age, the histological type of the tumor, the location of the tumor, the size of the tumor, the clinical lymph node stage by CT, and the clinical lymph node stage by PET-CT, without the use of a nomogram.

In Equation 1 or 2, p represents the predicted probability of mediastinal lymph node metastasis (N2-3). I represents an indicator variable for an individual clinical factor, and is assigned a value of 1 if the condition in parentheses is met, and 0 if it is not met. For example, if the individual's age is 68, in Equation 1 or 2, I (Age<60) is assigned a value of 0, and I (60≤Age<70) is assigned a value of 1. For example, if the histological type of the lung cancer tumor is adenocarcinoma, I (Histology=Adenocarcinoma) is 1, and I (Histology=Other non-squamous carcinoma) is 0.

In an embodiment, the probability of mediastinal lymph node metastasis in the lung cancer may be predicted or calculated by Equation 1 or Equation 2. Alternatively, a comprehensive prediction may be made by integrating the results from both Equation 1 and Equation 2.

In an embodiment, the method of predicting mediastinal lymph node metastasis in non-small cell lung cancer according to the present disclosure may further include classifying an individual into a low-risk group and a moderate-to-high risk group for mediastinal lymph node metastasis, based on the incidence rate of mediastinal lymph node metastasis. Specifically, the individual may be classified into a low-risk group and a moderate-to-high risk group for mediastinal lymph node metastasis based on the individual's calculated incidence rate of mediastinal lymph node metastasis according to the method. More specifically, for the low-risk group for mediastinal lymph node metastasis, invasive mediastinal biopsy, such as EBUS-TBNA, may not be recommended.

The method of predicting the occurrence of mediastinal lymph node metastasis was developed based on the clinical variables of patients who underwent EBUS-TBNA and/or surgery through a prospective cohort study. Therefore, the method can be useful in supporting decision-making regarding treatment methods for lung cancer patients, specifically non-small cell lung cancer patients, without involving an invasive staging step. If mediastinal lymph node metastasis is confirmed in a lung cancer patient, specifically a non-small cell lung cancer patient, primary surgical resection is not recommended. To confirm the presence or absence of mediastinal lymph node metastasis, invasive staging is recommended. Generally, the invasive staging step is done by EBUS-TBNA. This may be a disadvantage because it can be burdensome to the patient due to the invasive method, but according to the above method, it is possible to predict the occurrence of mediastinal lymph node metastasis without involving an invasive staging step such as EBUS-TBNA.

The present disclosure may be usefully used to determine the presence or absence of mediastinal metastasis in non-small cell lung cancer patients without performing invasive staging. Specifically, when a prediction model is constructed by considering information on a patient's age, tumor histology, tumor location, tumor size, clinical lymph node stage by CT, and clinical lymph node stage by PET-CT as variables, the present disclosure has a high area under the receiver operating characteristic curve (AUC) in predicting the presence or absence of mediastinal lymph node metastasis in lung cancer, and thus can be effectively used for decision-making on patient staging and treatment methods.

The method may be configured to provide information on the presence or absence of mediastinal lymph node metastasis in lung cancer based on information on the patient's age, histological type, tumor location, tumor size, clinical lymph node stage by CT, and clinical lymph node stage by PET-CT without the use of a nomogram.

Another aspect of the present disclosure provides an apparatus for predicting mediastinal metastasis of lung cancer.

The apparatus may include: (i) an input unit configured to receive information regarding a lung cancer patient's age, a histological type of a tumor, a location of the tumor, a size of the tumor, a clinical lymph node stage determined by CT, and a clinical lymph node stage determined by PET-CT;
(ii) a calculation unit configured to calculate a mediastinal lymph node metastasis prediction score for each of the information received by the input unit; and
(iii) a probability calculation unit configured to calculate a probability of mediastinal lymph node metastasis in the lung cancer from a total score, wherein the total score is a sum of all the calculated prediction scores.

The apparatus may further include an output unit configured to output a result of the calculation of the probability of mediastinal lymph node metastasis in the lung cancer. The output unit may be connected to at least one of the input unit, the calculation unit, and the probability calculation unit.

The output unit may output a nomogram to which the variables are matched.

The apparatus may be an apparatus for driving a web page, an application, or the like, and may include, for example, a computing device, a mobile device, a server, or the like. The apparatus may include components of a processor, a storage unit, a memory, a receiver, and an output unit, and the input unit, the calculation unit, and the probability calculation unit may be implemented through the components of the apparatus. When implemented as a server, the mediastinal lymph node metastasis prediction apparatus may be driven to transmit the calculated values to another device having an output unit.

Specifically, variables for the patient's age, the histological type of the lung cancer, the location of the tumor, the size of the tumor, the clinical lymph node stage by CT, and the clinical lymph node stage by PET-CT are input into the input unit.

In an embodiment, an output unit may be connected to the input unit, and the output unit may visually output a nomogram to which variables are matched and/or a predicted probability.

The calculation unit may calculate a prediction score for the variable input from the input unit. Specifically, the calculation unit may match a predetermined value to each of the variables. For example, the matching may be performed using a nomogram in which a range of measured values for each variable is matched to at least a portion of a score line having a minimum value and a maximum value.

The probability calculation unit receives the prediction score for each variable matched by the calculation unit and, based on this, calculates the probability of mediastinal lymph node metastasis for a lung cancer patient, specifically a non-small cell lung cancer patient. Specifically, the probability calculation unit may obtain a total score by summing all prediction scores for the variables, and may match the total score with a predetermined probability of mediastinal lymph node metastasis.

In an embodiment, the calculation unit and the probability calculation unit may use a PLUS-M nomogram, a PLUS-E nomogram, or both, shown below.

The nomogram may be determined by an equation for calculating the probability of mediastinal lymph node metastasis in lung cancer, specifically non-small cell lung cancer. For example, it may be expressed as in Equation 1 or Equation 2 below.

$$ln ( \frac{p}{1 - p} ) = - 5.2087 + 1.39 \; 18 \times I ( Age < 60 ) + 0.8888 \times I ( 60 \leq Age < 70 ) + 1.3 \; 945 \times I ( Histology = Adenocarcinoma ) + 1.3489 \times I ( Histology = Other non - squamous carcinoma ) + 0.517 \times I ( Location = central ) + 0.8184 \times I ( 3 cm < Tumor size \leq 5 cm ) + 0.4647 \times I ( Tumor size > 5 cm ) + 1.224 \times I ( cN by CT = N 1 ) + 1.74 \; 89 \times I ( cN by CT = N 2 or 3 ) + 1.6629 \times I ( cN by PET / CT = N 1 ) + 2.4 \; 198 \times I ( cN by PET / CT = N 2 or 3 ) \qquad [ Equation 1 ]$$

$$ln ( \frac{p}{1 - p} ) = - 5.4838 + 1.25 \; 38 \times I ( Age < 60 ) + 0.6143 \times I ( 60 \leq Age < 70 ) + 1.3 \; 333 \times I ( Histology = Adenocarcinoma ) + 1.0183 \times I ( Histology = Other non - squamous carcinoma ) + 0.3006 \times I ( Location = central ) + 0.7679 \times I ( 3 cm < Tumor size \leq 5 cm ) + 0.4159 \times I ( Tumor size > 5 cm ) + 1.2494 \times I ( cN by CT = N 1 ) + 2.02 \; 04 \times I ( cN by CT = N 2 or 3 ) + 1.7475 \times I ( cN by PET / CT = N 1 ) + 2.5 \; 994 \times I ( cN by PET / CT = N 2 or 3 ) \qquad [ Equation 2 ]$$

In the above equations, p represents the predicted probability of N2-3, and I represents an indicator variable for an individual clinical factor. The description of the indicator variable is as described above.

The present disclosure provides an apparatus for predicting mediastinal lymph node metastasis in lung cancer, the apparatus including: (i) an input unit configured to receive information regarding a lung cancer patient's age, a histological type of a tumor, a location of the tumor, a size of the tumor, a clinical lymph node stage determined by CT, and a clinical lymph node stage determined by PET-CT;
(ii) a probability calculation unit configured to calculate a probability of mediastinal lymph node metastasis in the lung cancer based on the information received by the input unit, according to Equation 1, Equation 2, or both.

$$ln ( \frac{p}{1 - p} ) = - 5.2087 + 1.39 \; 18 \times I ( Age < 60 ) + 0.8888 \times I ( 60 \leq Age < 70 ) + 1.3 \; 945 \times I ( Histology = Adenocarcinoma ) + 1.3489 \times I ( Histology = Other non - squamous carcinoma ) + 0.517 \times I ( Location = central ) + 0.8184 \times I ( 3 cm < Tumor size \leq 5 cm ) + 0.4647 \times I ( Tumor size > 5 cm ) + 1.224 \times I ( cN by CT = N 1 ) + 1.74 \; 89 \times I ( cN by CT = N 2 or 3 ) + 1.6629 \times I ( cN by PET / CT = N 1 ) + 2.4 \; 198 \times I ( cN by PET / CT = N 2 or 3 ) \qquad [ Equation 1 ]$$

$$ln ( \frac{p}{1 - p} ) = - 5.4838 + 1.25 \; 38 \times I ( Age < 60 ) + 0.6143 \times I ( 60 \leq Age < 70 ) + 1.3 \; 333 \times I ( Histology = Adenocarcinoma ) + 1.0183 \times I ( Histology = Other non - squamous carcinoma ) + 0.3006 \times I ( Location = central ) + 0.7679 \times I ( 3 cm < Tumor size \leq 5 cm ) + 0.4159 \times I ( Tumor size > 5 cm ) + 1.2494 \times I ( cN by CT = N 1 ) + 2.02 \; 04 \times I ( cN by CT = N 2 or 3 ) + 1.7475 \times I ( cN by PET / CT = N 1 ) + 2.5 \; 994 \times I ( cN by PET / CT = N 2 or 3 ) \qquad [ Equation 2 ]$$

In the above equations, p represents the predicted probability of N2-3, and I represents an indicator variable for an individual clinical factor.

The apparatus may further include an output unit configured to output a result of the calculation of the probability of mediastinal lymph node metastasis in the lung cancer. The output unit may be connected to at least one of the input unit and the probability calculation unit.

The apparatus may be an apparatus for driving a web page, an application, or the like, and may include, for example, a computing device, a mobile device, a server, or the like. The apparatus may include components of a processor, a storage unit, a memory, a receiver, and an output unit, and the input unit and the probability calculation unit may be implemented through the components of the apparatus. When implemented as a server, the mediastinal lymph node metastasis prediction apparatus may be driven to transmit the calculated values to another device having an output unit.

In an embodiment, the apparatus for predicting mediastinal lymph node metastasis in lung cancer according to the present disclosure may further include a component for classifying into a non-risk group, a risk group, a high-risk group, and a very-high-risk group for mediastinal lymph node metastasis, according to the incidence rate of mediastinal lymph node metastasis. A known algorithm for classifying a patient's risk group may be used in the apparatus, but it is not limited thereto, and various other algorithms may be considered.

### BRIEF DESCRIPTION OF THE DRAWINGS

The above and other aspects, features, and advantages of certain embodiments of the disclosure will be more apparent from the following description taken in conjunction with the accompanying drawings in which:

FIG. 1 illustrates an exemplary nomogram according to an aspect of the present disclosure.

FIG. 2 is a graph showing receiver operating characteristic (ROC) curves for a mediastinal lymph node metastasis prediction model according to an aspect of the present disclosure. (Development Cohort; and Validation Cohort).

FIG. 3 is a calibration plot for verifying the predictive ability of a mediastinal lymph node metastasis prediction model according to an aspect of the present disclosure (Observed Risk; Predicted Risk; Development Cohort; and Validation Cohort).

### DETAILED DESCRIPTION

The respective features of various embodiments of the present disclosure may be partially or wholly combined or combined with each other, and, as will be fully understood by those skilled in the art, various technical interoperations and operations are possible, and each embodiment may be implemented independently of each other, or may be implemented together in an associated relationship.

In interpreting the components, even if there is no separate explicit description, it is to be interpreted as including an error range. In addition, the prediction model for mediastinal lymph node metastasis in non-small cell lung cancer may be updated by integrating it with prospective cohort data in the future to ensure generalization and robustness of the estimates.

Hereinafter, it will be described in more detail through Examples. However, these Examples are for illustrative purposes of one or more specific embodiments, and the scope of the present disclosure is not limited to these Examples.

### Example 1. Study Population

In this Example, a prospective cohort study was conducted at five hospitals in Korea (National Cancer Center [NCC], Seoul National University Bundang Hospital [SNUBH], Asan Medical Center [AMC], Samsung Medical Center [SMC], and Seoul National University Hospital [SNUH]). Specifically, 600 patients (aged 18 to 80 years) with potentially operable non-small cell lung cancer (NSCLC) were enrolled from July 2016 to June 2019. Based on previous studies, the sample size was calculated to have an AUC value of 0.8 with a width of 0.08 (95% confidence interval) and an N2-3 disease prevalence of 35%. Subsolid nodular tumors containing a solid portion of 1 cm or less in diameter and solid nodular tumors with cT1aN0 on CT and PET-CT were excluded. Contrast-enhanced chest CT and PET-CT scans were performed within 30 days prior to performing EBUS-TBNA. This study was approved by the institutional review board of NCC (Identifiers: NCC-2016-0156 and 2021-0307) and other hospitals (Seoul National University Bundang Hospital (B-1608-360-301), Asan Medical Center (2016-0713), Samsung Medical Center (2016-07-125-003), and Seoul National University Hospital (1608-006-784)), and informed consent was obtained from all participants in the development cohort prior to enrollment.

The general characteristics of the 600 patients were as follows. Of the 600 enrolled patients, 589 (NCC=459, SNUBH=52, AMC=42, SMC=27, and SNUH=9) were included in the development cohort. A validation cohort of 309 patients was retrospectively recruited separately from the development cohort. The basic characteristics of the development and validation cohorts were similar, except for the use of EUS-B-FNA (endoscopic ultrasound with bronchoscope-guided fine needle aspiration) and the number of examined lymph node stations and the number of aspirations of EBUS-TBNA. In the development cohort, the prevalence of N2-3 stage was 35.3%, and the sensitivity of EBUS-TBNA was 87.0%, and in the validation cohort, they were 36.6% and 81.4%, respectively.

### Example 2. EBUS-TBNA and Clinical Data Collection

### 2.1. EBUS-TBNA

In the development cohort, EBUS-TBNA was performed by six experienced bronchoscopists under conscious sedation. Target lymph nodes (LNs) included abnormal lymph nodes on CT or PET-CT that were accessible by EBUS-TBNA, and were determined according to the bronchoscopist's judgment, considering the possibility of lymph node metastasis and its impact on treatment. In cases of lymph nodes that were difficult to access by EBUS-TBNA, aspiration was performed using EUS-B-FNA if accessible. The classification of cN (clinical N) stage determined by EBUS-TBNA included the results of EUS-B-FNA. Within 30 days after EBUS-TBNA, surgery with systematic LN dissection was recommended for patients with cN0-1 by EBUS-TBNA.

### 2.2. Collection of Patient Clinical Information

Data on age, sex, tumor histology, tumor location (central or peripheral), clinical tumor stage (cT) according to tumor size confirmed on axial chest CT, and clinical lymph node stage (cN) by CT, PET-CT, and EBUS-TBNA were collected.

Age was categorized as less than 60 years, 60 to 70 years, and 70 years or older. Histological type was classified into three groups: squamous cell carcinoma, adenocarcinoma (including non-small cell lung cancer with an adenocarcinoma component), and others. A central tumor was defined as a tumor located in the inner one-third of the hemithorax based on the innermost part of the tumor on CT. The lines dividing the hemithorax into thirds were drawn as concentric circles originating from the midline. The staging criteria for lung cancer were based on the 8th edition of the International Association for the Study of Lung Cancer staging criteria. For surgical cases, the pathological N (pN) stage was reviewed. For patients with pNx (regional lymph node metastasis cannot be assessed) in whom systematic lymph node dissection was not performed during surgery, a follow-up investigation of at least 12 months was performed to identify positive mediastinal lymph node metastasis.

### Example 3. Construction of a Nomogram for Predicting Mediastinal Lymph Node Metastasis

Statistical analysis was performed on the patients in Example 1 to predict the presence of mediastinal lymph node metastasis using clinical information from potentially operable non-small cell lung cancer patients.

The prediction model for lung cancer staging-mediastinal metastasis is abbreviated as PLUS-M. The outcome for the development of the PLUS-M model is N2 or N3 lymph node metastasis confirmed by EBUS-TBNA or surgical lymph node dissection. Another prediction model, the prediction model for lung cancer staging-mediastinal metastasis detection by EBUS-TBNA, is abbreviated as PLUS-E. The outcome for the development of the PLUS-E model is N2 or N3 lymph node metastasis diagnosed by EBUS-TBNA.

Specifically, univariable logistic regression analysis was performed on the variables of age, sex, tumor histology, tumor location, clinical tumor stage (cT) according to tumor size, clinical lymph node metastasis stage (cN) by CT, and clinical lymph node metastasis stage (cN) by PET-CT. Variables included in the final prediction model were determined by a backward selection method, in which all variables with p<0.2 were included in the multivariable analysis, and variables with p>0.05 were eliminated. Significant risk factors in PLUS-M were included in PLUS-E. Regression analysis results were expressed as odds ratios (OR) with 95% confidence intervals (CI) and p-values.

Accordingly, clinical nomograms were constructed for PLUS-M and PLUS-E, and the probability of mediastinal lymph node metastasis predicted by PLUS-M and PLUS-E was calculated based on risk factors. All statistical analyses were performed using SAS 9.4 (SAS Institute Inc., Cary, NC, USA) and R project software (version 4.1.1).

Table 1 shows the results of the univariable and multivariable regression analyses of the mediastinal lymph node metastasis prediction model, PLUS-M.

TABLE 1

| | (−)N2-3 | | (+)N2-3 | | | | | | | |
|---|---|---|---|---|---|---|---|---|---|---|
| | (n = 381) | | (n = 208) | | Univariable | | | Multivariable | | |
| Variable | n (%) | | n (%) | | 95% CI | | p-value | 95% CI | | p-value |
| Age, ≥70 | 153 | (40.2) | 59 | (28.4) | 1 | | (0.016) | 1 | | (<0.001) |
| Age, 60-70 | 136 | (35.7) | 85 | (40.9) | 1.62 | (1.08-2.43) | 0.019 | 2.43 | (1.43-4.13) | 0.001 |
| Age, <60 | 92 | (24.1) | 64 | (30.8) | 1.80 | (1.16-2.80) | 0.008 | 4.02 | (2.16-7.49) | <0.001 |
| Sex, female | 153 | (40.2) | 64 | (30.8) | 1 | | | | | |
| Sex, male | 228 | (59.8) | 144 | (69.2) | 1.51 | (1.06-2.16) | 0.024 | | | |
| Histologic | 103 | (27.0) | 59 | (28.4) | 1 | | (0.040) | 1 | | (<0.001) |
| type, | | | | | | | | | | |
| Squamous | | | | | | | | | | |
| cell | | | | | | | | | | |
| carcinoma | | | | | | | | | | |
| Histologic | 265 | (69.6) | 132 | (63.5) | 0.87 | (0.59-1.27) | 0.474 | 4.03 | (2.27-7.18) | <0.001 |
| type, | | | | | | | | | | |
| Adenocarcinoma | | | | | | | | | | |
| Histologic | 13 | (3.4) | 17 | (8.2) | 2.28 | (1.04-5.03) | 0.041 | 3.85 | (1.39-10.66) | 0.009 |
| type, Others | | | | | | | | | | |
| Tumor | 232 | (60.9) | 77 | (37.0) | 1 | | | 1 | | |
| location, | | | | | | | | | | |
| Peripheral | | | | | | | | | | |
| Tumor | 149 | (39.1) | 131 | (63.0) | 2.65 | (1.87-3.75) | <0.001 | 1.68 | (1.02-2.76) | 0.042 |
| location, | | | | | | | | | | |
| Central | | | | | | | | | | |
| cT stage by | 227 | (59.6) | 67 | (32.2) | 1 | | (<0.001) | 1 | | (0.009) |
| tumor size, | | | | | | | | | | |
| cT1 | | | | | | | | | | |
| cT stage by | 113 | (29.7) | 97 | (46.6) | 2.91 | (1.98-4.27) | <0.001 | 2.27 | (1.34-3.83) | 0.002 |
| tumor size, | | | | | | | | | | |
| cT2 | | | | | | | | | | |
| cT stage by | 41 | (10.8) | 44 | (21.2) | 3.64 | (2.19-6.03) | <0.001 | 1.59 | (0.78-3.26) | 0.204 |
| tumor size, | | | | | | | | | | |
| cT3-4 | | | | | | | | | | |
| cN stage by | 276 | (72.4) | 44 | (21.2) | 1 | | (<0.001) | 1 | | (<0.001) |
| CT, cN0 | | | | | | | | | | |
| cN stage by | 37 | (9.7) | 30 | (14.4) | 5.09 | (2.86-9.06) | <0.001 | 3.40 | (1.63-7.11) | 0.001 |
| CT, cN1 | | | | | | | | | | |
| cN stage by | 68 | (17.9) | 134 | (64.4) | 12.36 | (8.03-19.03) | <0.001 | 5.75 | (3.15-10.48) | <0.001 |
| CT, cN2-3 | | | | | | | | | | |
| cN stage by | 253 | (66.4) | 29 | (13.9) | 1 | | (<0.001) | 1 | | (<0.001) |
| PET-CT, cN0 | | | | | | | | | | |
| cN stage by | 41 | (10.8) | 25 | (12.0) | 5.32 | (2.84-9.97) | <0.001 | 5.27 | (2.43-11.46) | <0.001 |
| PET-CT, cN1 | | | | | | | | | | |
| cN stage by | 87 | (22.8) | 154 | (74.0) | 15.44 | (9.69-24.60) | <0.001 | 11.24 | (6.14-20.58) | <0.001 |
| PET-CT, cN2-3 | | | | | | | | | | |

As shown in Table 1, in the regression analysis for PLUS-M, univariable analysis confirmed that younger age (<60 and 60-70), male sex, other histological type, central tumor location, higher tumor stage (cT2, cT3-4), cN1 or cN2-3 stage on CT, and cN1 or cN2-3 stage on PET-CT were statistically significant risk factors for mediastinal lymph node metastasis. Multivariable analysis confirmed that younger age (<60 and 60-70), adenocarcinoma or other histological type, central tumor location, clinical tumor stage cT2, cN1 or cN2-3 stage on CT, and cN1 or cN2-3 stage on PET-CT were risk factors for mediastinal lymph node metastasis, and male sex was not a risk factor (OR=1.08, 95% confidence interval (95% CI)=0.63-1.87, p=0.779).

Table 2 shows the results of the univariable and multivariable regression analyses of the mediastinal lymph node metastasis prediction model, PLUS-E.

TABLE 2

| | (−)N2-3 | | (+)N2-3 | | | | | | | |
|---|---|---|---|---|---|---|---|---|---|---|
| | (n = 408) | | (n = 181) | | Univariable | | | Multivariable | | |
| Variable | n (%) | | n (%) | | 95% CI | | p-value | 95% CI | | p-value |
| Age, ≥70 | 159 | (39.0) | 53 | (29.3) | 1 | | (0.063) | 1 | | (<0.001) |
| Age, 60-70 | 149 | (36.5) | 72 | (39.8) | 1.45 | (0.95-2.20) | 0.083 | 1.85 | (1.07-3.19) | 0.027 |
| Age, <60 | 100 | (24.5) | 56 | (30.9) | 1.68 | (1.07-2.64) | 0.024 | 3.50 | (1.84-6.69) | <0.001 |
| Sex, female | 162 | (39.7) | 55 | (30.4) | 1 | | | | | |
| Sex, male | 246 | (60.3) | 126 | (69.6) | 1.51 | (1.04-2.19) | 0.031 | | | |
| Histologic type, | 109 | (26.7) | 53 | (29.3) | 1 | | (0.105) | 1 | | (<0.001) |
| Squamous cell | | | | | | | | | | |
| carcinoma | | | | | | | | | | |
| Histologic type, | 283 | (69.4) | 114 | (63.0) | 0.83 | (0.56-1.23) | 0.349 | 3.79 | (2.11-6.82) | <0.001 |
| Adenocarcinoma | | | | | | | | | | |
| Histologic type, | 16 | (3.9) | 14 | (7.7) | 1.80 | (0.82-3.96) | 0.144 | 2.77 | (1.01-7.60) | 0.048 |
| Others | | | | | | | | | | |
| Tumor location, | 241 | (59.1) | 68 | (37.6) | 1 | | | 1 | | |
| Peripheral | | | | | | | | | | |
| Tumor location, | 167 | (40.9) | 113 | (62.4) | 2.40 | (1.67-3.44) | <0.001 | 1.35 | (0.80-2.28) | 0.262 |
| Central | | | | | | | | | | |
| cT stage by | 237 | (58.1) | 57 | (31.5) | 1 | | (<0.001) | 1 | | (0.025) |
| tumor size, cT1 | | | | | | | | | | |
| cT stage by | 125 | (30.6) | 85 | (47.0) | 2.83 | (1.90-4.22) | <0.001 | 2.16 | (1.23-3.76) | 0.007 |
| tumor size, cT2 | | | | | | | | | | |
| cT stage by | 46 | (11.3) | 39 | (21.5) | 3.53 | (2.11-5.90) | <0.001 | 1.52 | (0.73-3.17) | 0.269 |
| tumor size, cT3-4 | | | | | | | | | | |
| cN stage by CT, cN0 | 290 | (71.1) | 30 | (16.6) | 1 | | (<0.001) | 1 | | (<0.001) |
| cN stage by CT, cN1 | 43 | (10.5) | 24 | (13.3) | 5.40 | (2.89-10.08) | <0.001 | 3.49 | (1.60-7.61) | 0.002 |
| cN stage by CT, cN2-3 | 75 | (18.4) | 127 | (70.2) | 16.37 | (10.21-26.24) | <0.001 | 7.54 | (4.01-14.18) | <0.001 |
| cN stage by PET- | 264 | (64.7) | 18 | (9.9) | 1 | | (<0.001) | 1 | | (<0.001) |
| CT, cN0 | | | | | | | | | | |
| cN stage by PET- | 47 | (11.5) | 19 | (10.5) | 5.93 | (2.90-12.13) | <0.001 | 5.74 | (2.43-13.56) | <0.001 |
| CT, cN1 | | | | | | | | | | |
| cN stage by PET- | 97 | (23.8) | 144 | (79.6) | 21.77 | (12.66-37.45) | <0.001 | 13.46 | (6.95-26.04) | <0.001 |
| CT, cN2-3 | | | | | | | | | | |

As shown in Table 2, in the regression analysis for PLUS-E, univariable analysis confirmed that younger age (<60), male sex, central tumor location, higher tumor stage (cT2, cT3-4), cN1 or cN2-3 stage on CT, and cN1 or cN2-3 stage on PET-CT were statistically significant risk factors for mediastinal lymph node metastasis. Multivariable analysis confirmed that younger age (<60 and 60-70), adenocarcinoma or other histological type, clinical tumor stage cT2, cN1 or cN2-3 stage on CT, and cN1 or cN2-3 stage on PET-CT were statistically significant risk factors for mediastinal lymph node metastasis. Tumor location (p=0.262) was not found to be a significant risk factor in the multivariable analysis, but was included as a risk factor to maintain consistency with PLUS-M. Also, male sex was not found to be a risk factor (OR=1.02, 95% confidence interval (95% CI)=0.57-1.82, p=0.953).

Based on the above results, a nomogram was constructed by setting six variables—patient age, tumor histological type, tumor location, tumor size, clinical lymph node stage by CT, and clinical lymph node stage by PET-CT—as risk factors for predicting mediastinal lymph node metastasis in lung cancer, specifically non-small cell lung cancer.

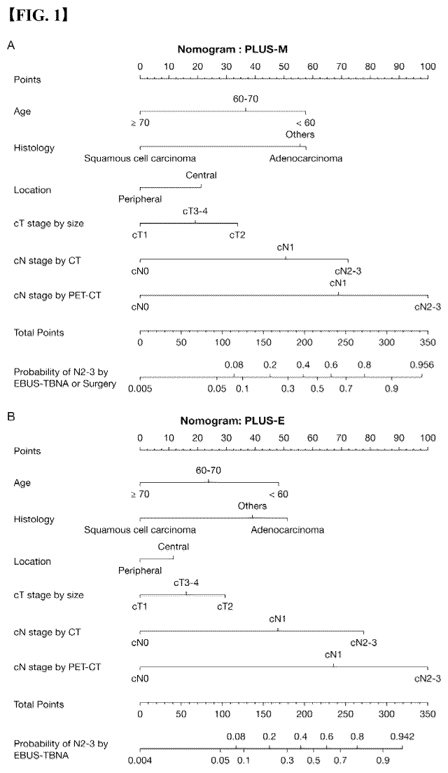

FIG. 1 exemplarily illustrates a nomogram according to an aspect.

### Example 4. Verification of the Nomogram for Predicting Mediastinal Lymph Node Metastasis

To verify whether the nomogram established as above can accurately predict the presence of cancer metastasis in mediastinal lymph nodes in patients with non-small cell lung cancer, verification was performed as follows.

### 4.1. Prediction Model Validation and Assessment

For model validation, a retrospective cohort study was conducted at the National Cancer Center during a different period (June 2019 to August 2021) based on the same criteria and imaging data.

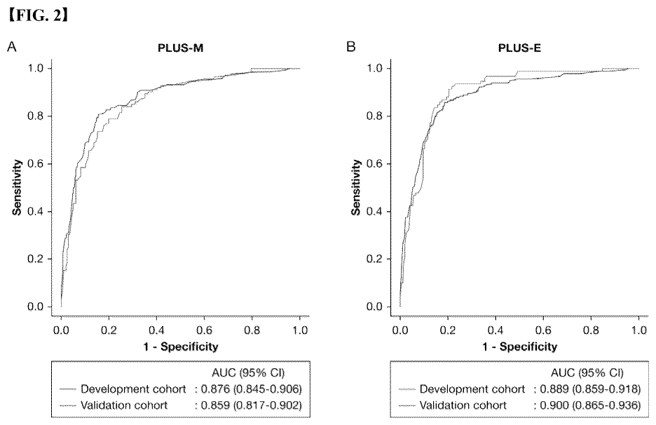

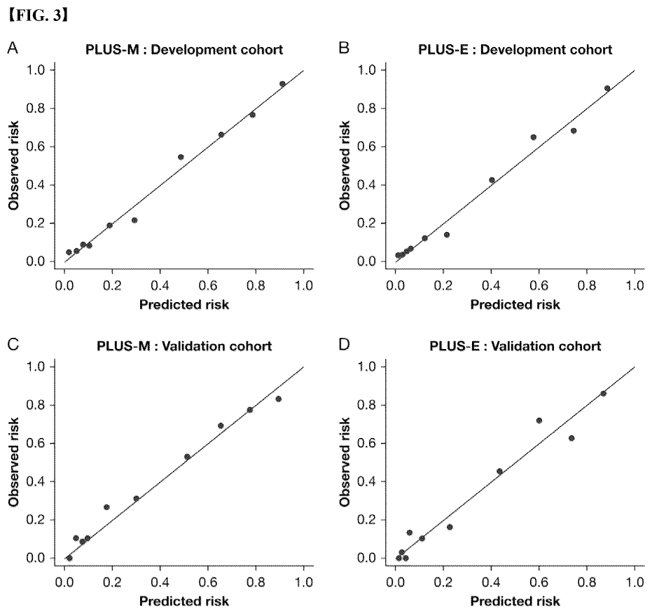

For discrimination, ROC curves for each model are shown in FIG. 2, and AUCs were calculated and are shown in Table 3. PLUS-M and PLUS-E were applied to the data of the validation cohort, ROC curves are presented, and AUCs were calculated. Internal validation was performed by calculating optimism-adjusted AUCs using 1000 bootstrap samples. Calibration was assessed using the Hosmer-Lemeshow test and the Brier score, which are shown in Table 3. Calibration plots (observed vs. predicted) were created by dividing the predicted risk into deciles and are shown in FIG. 3.

Table 3 shows the results of evaluating and validating the PLUS-M and PLUS-E prediction models.

TABLE 3

| | Characteristic | | PLUS-M | | | PLUS-E | | |
|---|---|---|---|---|---|---|---|---|
| | | Sensitivity | | Hosmer- | | | Hosmer- | |
| | Prevalence | of EBUS- | AUC | Lemes | | AUC | Lemes | |
| | of N2- | TBNA | (95% | how p | Brier | (95% | how p | Brier |
| | 3%(n/n) | %(n/n) | CI) | Value | Score | CI) | Value | Score |
| Development | 35.3 | 87.0 | 0.876 | 0.658 | 0.129 | 0.889 | 0.569 | 0.118 |
| cohort | (208/589) | (181/208) | (0.845- | | | (0.859- | | |
| (n = 589) | | | 0.906) | | | 0.918) | | |
| Validation | 36.6 | 81.4 | 0.859 | 0.609 | 0.144 | 0.900 | 0.361 | 0.112 |
| cohort | (113/309) | (92/113) | (0.817- | | | (0.865- | | |
| (n = 309) | | | 0.902) | | | 0.936) | | |

As shown in Table 3, model fit was confirmed in the development cohort for PLUS-M (Hosmer-Lemeshow P=0.658, Brier score=0.129) and PLUS-E (Hosmer-Lemeshow P=0.569, Brier score=0.118). Model fit was confirmed in the validation cohort for PLUS-M (Hosmer-Lemeshow P=0.609, Brier score=0.144) and PLUS-E (Hosmer-Lemeshow P=0.361, Brier score=0.112).

FIG. 2 is a graph showing ROC curves for the mediastinal lymph node metastasis prediction model according to an aspect. Development Cohort: development cohort; Validation Cohort: validation cohort.

FIG. 3 is a calibration plot for verifying the predictive ability of the mediastinal lymph node metastasis prediction model according to an aspect. Observed risk: observed risk; Predicted Risk: predicted risk; Development Cohort: development cohort; Validation Cohort: validation cohort.

As shown in FIG. 2, in the development cohort, the AUCs of PLUS-M and PLUS-E were 0.876 (95% CI, 0.845-0.906) and 0.889 (95% CI, 0.859-0.918), respectively, and the optimism-adjusted AUCs were 0.866 and 0.879, respectively. In the validation cohort, the AUCs of PLUS-M and PLUS-E were 0.859 (95% CI, 0.817-0.902) and 0.900 (95% CI, 0.865-0.936), respectively.

### Example 5. Application of the Prediction Model Using Probability Thresholds

Subjects for EBUS-TBNA were selected in the development and validation cohorts based on the recommendations of the European Society of Thoracic Surgeons (ESTS), the modified American College of Chest Physicians (ACCP) (cN1-3 stage or central lung cancer on CT or PET-CT), the National Institute for Health and Care Excellence (NICE) guidelines, and various probability thresholds (≥10%, 8%, or 5%) of PLUS-M and PLUS-E. The sensitivity of the model/guideline recommendations for N2-3 stage, the N2-3 confirmation rate by EBUS-TBNA, unexpected N2-3 after surgery, and the EBUS-TBNA prevention rate were calculated.

Table 4 analyzes the expected diagnostic outcomes after applying different guidelines/models or various probability thresholds of PLUS-M and PLUS-E within the development cohort.

TABLE 4

| | | Negative | | | |
|---|---|---|---|---|---|
| | | Predictive | Detection | | Prevented |
| | Sensitivity of | Value of | of N2-3 | | EBUS-TBNA |
| | Model/Guidelines | N2-3 | Disease by | | by |
| | for N2-3 | Disease | EBUS- | Unforeseen | Development |
| EBUS-TBNA | Disease | % (95% | TBNA | N2-3 | cohort |
| Staging Groups | % (95% CI) | CI) | % (95% CI) | % (95% CI) | % (95% CI) |
| Development | — | — | 87.0 | 6.6 | 0 |
| cohort, | | | (81.7-91.3) | (4.4-9.5) | |
| n = 589 | | | | | |
| ESTS guidelines, | 96.6 | 94.7 | 84.6 | 7.7 | 22.6 |
| n = 456 | (93.2-98.6) | (89.5-97.9) | (79.0-89.2) | (5.4-10.8) | (19.3-26.2) |
| Modified CHEST | 95.2 | 93.9 | 84.1 | 8.0 | 27.8 |
| guidelines | (91.3-97.7) | (89.1-97.0) | (78.4-88.8) | (5.6-11.0) | (24.3-31.7) |
| (modified) | | | | | |
| n = 425 | | | | | |
| NICE guidelines, | 88.9 | 90.4 | 80.8 | 9.5 | 40.6 |
| n = 350 | (83.9-92.9) | (85.9-93.8) | (74.7-85.9) | (6.9-12.7) | (36.6-44.7) |
| PLUS-M | 93.3 | 93.2 | 83.2 | 8.4 | 35.0 |
| probability ≥10% | (89.0-96.3) | (88.9-96.2) | (77.4-88.0) | (5.9-11.5) | (31.1-39.0) |
| n = 383 | | | | | |
| PLUS-M | 95.7 | 93.5 | 84.1 | 8.0 | 23.4 |
| probability ≥ 8% | (91.9-98.0) | (88.0-97.0) | (78.4-88.8) | (5.6-11.0) | (20.1-27.1) |
| n = 451 | | | | | |
| PLUS-M | 98.6 | 95.8 | 86.1 | 7.1 | 12.2 |
| probability ≥ 5% | (95.8-99.7) | (88.3-99.1) | (80.6-90.5) | (4.8-10.0) | (9.7-15.1) |
| n = 517 | | | | | |
| PLUS-E | 91.3 | 92.7 | 81.7 | 9.1 | 41.9 |
| probability ≥ | (86.7-94.8) | (88.7-95.6) | (75.8-86.7) | (6.5-12.2) | (37.9-46.0) |
| 10% | | | | | |
| n = 342 | | | | | |
| PLUS-E | 91.3 | 92.4 | 81.7 | 9.1 | 40.4 |
| probability ≥ 8% | (86.7-94.8) | (88.3-95.5) | (75.8-86.7) | (6.5-12.2) | (36.4-44.5) |
| n = 351 | | | | | |
| PLUS-E | 94.7 | 92.3 | 84.1 | 8.0 | 24.1 |
| probability ≥ 5% | (90.7-97.3) | (86.6-96.1) | (78.4-88.8) | (5.6-11.0) | (20.7-27.8) |
| n = 447 | | | | | |
| PLUS-M | 93.3 | 93.2 | 83.2 | 8.4 | 35.0 |
| probability ≥ | (89.0-96.3) | (88.9-96.2) | (77.4-88.0) | (5.9-11.5) | (31.1-39.0) |
| 10% and | | | | | |
| PLUS-E | | | | | |
| probability ≥ 5% | | | | | |
| n = 383 | | | | | |

As shown in Table 4, when calculated based on ESTS, the N2-3 stage detection sensitivity, N2-3 stage detection rate of EBUS-TBNA, unexpected N2-3 detection rate during surgery, and the rate of prevented EBUS-TBNA procedures for each different guideline/model were 96.6%, 84.6%, 7.7%, and 22.6%, respectively. When a probability threshold of 10% was applied, the respective rates were 93.3%, 83.2%, 8.4%, and 35.0%. This prevented 73 more EBUS-TBNA procedures compared to ESTS. In addition, PLUS-E with a 10% probability threshold had a higher unexpected N2-3 detection rate (9.1%) than PLUS-M with the same probability threshold, and prevented more EBUS-TBNA procedures.

Based on the above results, in the models (PLUS-M, PLUS-E) for predicting mediastinal lymph node metastasis and detection by EBUS-TBNA in potentially operable non-small cell lung cancer patients, younger age, adenocarcinoma, other non-squamous cell histological types, central tumor location, tumor size (>3-5 cm), cN1 and cN2-3 stage on CT, and cN1 and cN2-3 stage on PET-CT were identified as risk factors for mediastinal lymph node metastasis in PLUS-M. Based on this, it was confirmed that it can be usefully used for staging and decision-making on treatment methods for non-small cell lung cancer patients, such as invasive mediastinal staging.

According to an aspect of the present disclosure, a method and apparatus for predicting mediastinal lymph node metastasis in non-small cell lung cancer provides information on the presence of mediastinal lymph node metastasis in potentially operable lung cancer patients, specifically non-small cell lung cancer patients. Therefore, the method and the apparatus can be usefully used for staging and decision-making on treatment methods for non-small cell lung cancer patients, such as invasive mediastinal staging.

What is claimed is:

1. A method for predicting mediastinal lymph node metastasis in lung cancer, the method comprising:
(a) obtaining information regarding a lung cancer patient's age, a histological type of a tumor, a location of the tumor, a size of the tumor, a clinical lymph node stage determined by CT, and a clinical lymph node stage determined by PET-CT;
(b) calculating a mediastinal lymph node metastasis prediction score for each variable obtained in step (a); and
(c) calculating a probability of mediastinal lymph node metastasis in the lung cancer based on a total score, wherein the total score is a sum of the prediction scores for all variables calculated in step (b).

2. The method of claim 1, wherein the calculating the mediastinal lymph node metastasis prediction score in step (b) comprises using a nomogram, wherein information for each variable is matched to at least a portion of a score line having a minimum value and a maximum value.

3. The method of claim 2, wherein the calculating the mediastinal lymph node metastasis prediction score in steps (b) and (c) comprises using a PLUS-M model nomogram, a PLUS-E model nomogram, or both.

[symbol]

4. A method for predicting mediastinal lymph node metastasis in lung cancer, the method comprising: (a-1) obtaining information regarding a lung cancer patient's age, a histological type of a tumor, a location of the tumor, a size of the tumor, a clinical lymph node stage determined by CT, and a clinical lymph node stage determined by PET-CT; and
(b-1) deriving a probability of mediastinal lymph node metastasis in the lung cancer based on the information obtained in step (a-1), according to Equation 1, Equation 2, or both,
$$ln ( \frac{p}{1 - p} ) = - 5.2087 + 1.39 \; 18 \times I ( Age < 60 ) + 0.8888 \times I ( 60 \leq Age < 70 ) + 1.3 \; 945 \times I ( Histology = Adenocarcinoma ) + 1.3489 \times I ( Histology = Other non - squamous carcinoma ) + 0.517 \times I ( Location = central ) + 0.8184 \times I ( 3 cm < Tumor size \leq 5 cm ) + 0.4647 \times I ( Tumor size > 5 cm ) + 1.224 \times I ( cN by CT = N 1 ) + 1.74 \; 89 \times I ( cN by CT = N 2 or 3 ) + 1.6629 \times I ( cN by PET / CT = N 1 ) + 2.4 \; 198 \times I ( cN by PET / CT = N 2 or 3 ) \qquad [ Equation 1 ]$$
$$ln ( \frac{p}{1 - p} ) = - 5.4838 + 1.25 \; 38 \times I ( Age < 60 ) + 0.6143 \times I ( 60 \leq Age < 70 ) + 1.3 \; 333 \times I ( Histology = Adenocarcinoma ) + 1.0183 \times I ( Histology = Other non - squamous carcinoma ) + 0.3006 \times I ( Location = central ) + 0.7679 \times I ( 3 cm < Tumor size \leq 5 cm ) + 0.4159 \times I ( Tumor size > 5 cm ) + 1.2494 \times I ( cN by CT = N 1 ) + 2.02 \; 04 \times I ( cN by CT = N 2 or 3 ) + 1.7475 \times I ( cN by PET / CT = N 1 ) + 2.5 \; 994 \times I ( cN by PET / CT = N 2 or 3 ) \qquad [ Equation 2 ]$$
wherein, in Equations 1 and 2, p represents a predicted probability of mediastinal lymph node metastasis (N2-3), and I represents an indicator variable for an individual clinical factor.

5. An apparatus for predicting mediastinal lymph node metastasis in lung cancer, the apparatus comprising: (i) an input unit configured to receive information regarding a lung cancer patient's age, a histological type of a tumor, a location of the tumor, a size of the tumor, a clinical lymph node stage determined by CT, and a clinical lymph node stage determined by PET-CT;
(ii) a calculation unit configured to calculate a mediastinal lymph node metastasis prediction score for each piece of the information received by the input unit; and
(iii) a probability calculation unit configured to calculate a probability of mediastinal lymph node metastasis in the lung cancer from a total score, wherein the total score is a sum of all the prediction scores calculated by the calculation unit.

6. The apparatus of claim 5, wherein the calculation unit is configured to calculate the mediastinal lymph node metastasis prediction score using a nomogram, wherein information for each variable is matched to at least a portion of a score line having a minimum value and a maximum value.

7. The apparatus of claim 6, wherein the nomogram used by the calculation unit and the probability calculation unit is a PLUS-M model nomogram, a PLUS-E model nomogram, or both.

[symbol]

8. An apparatus for predicting mediastinal lymph node metastasis in lung cancer, the apparatus comprising:
(i) an input unit configured to receive information regarding a lung cancer patient's age, a histological type of a tumor, a location of the tumor, a size of the tumor, a clinical lymph node stage determined by CT, and a clinical lymph node stage determined by PET-CT; and
(ii) a probability calculation unit configured to calculate a probability of mediastinal lymph node metastasis in the lung cancer based on the information received by the input unit, according to Equation 1, Equation 2, or both,
$$ln ( \frac{p}{1 - p} ) = - 5.2087 + 1.39 \; 18 \times I ( Age < 60 ) + 0.8888 \times I ( 60 \leq Age < 70 ) + 1.3 \; 945 \times I ( Histology = Adenocarcinoma ) + 1.3489 \times I ( Histology = Other non - squamous carcinoma ) + 0.517 \times I ( Location = central ) + 0.8184 \times I ( 3 cm < Tumor size \leq 5 cm ) + 0.4647 \times I ( Tumor size > 5 cm ) + 1.224 \times I ( cN by CT = N 1 ) + 1.74 \; 89 \times I ( cN by CT = N 2 or 3 ) + 1.6629 \times I ( cN by PET / CT = N 1 ) + 2.4 \; 198 \times I ( cN by PET / CT = N 2 or 3 ) \qquad [ Equation 1 ]$$
$$ln ( \frac{p}{1 - p} ) = - 5.4838 + 1.25 \; 38 \times I ( Age < 60 ) + 0.6143 \times I ( 60 \leq Age < 70 ) + 1.3 \; 333 \times I ( Histology = Adenocarcinoma ) + 1.0183 \times I ( Histology = Other non - squamous carcinoma ) + 0.3006 \times I ( Location = central ) + 0.7679 \times I ( 3 cm < Tumor size \leq 5 cm ) + 0.4159 \times I ( Tumor size > 5 cm ) + 1.2494 \times I ( cN by CT = N 1 ) + 2.02 \; 04 \times I ( cN by CT = N 2 or 3 ) + 1.7475 \times I ( cN by PET / CT = N 1 ) + 2.5 \; 994 \times I ( cN by PET / CT = N 2 or 3 ) \qquad [ Equation 2 ]$$
wherein, in Equations 1 and 2, p represents a predicted probability of mediastinal lymph node metastasis (N2-3), and I represents an indicator variable for an individual clinical factor.

9. The apparatus of claim 5, further comprising
an output unit configured to output a result of the calculation of the probability of mediastinal lymph node metastasis in the lung cancer.

10. The apparatus of claim 8, further comprising
an output unit configured to output a result of the calculation of the probability of mediastinal lymph node metastasis in the lung cancer.
